(19) United States
(12) Patent Application Publication
(10) Pub. No.: US 2009/0099555 A1
(43) Pub. Date: Apr. 16, 2009

(54) REDUCTION OF RF INDUCED TISSUE HEATING USING CONDUCTIVE SURFACE PATTERN

(76) Inventors: Ingmar Viohl, Canyon Country, CA (US); Bridget D. Viohl, Milwaukee, WI (US); Craig J. Peterson, Milwaukee, WI (US)

Correspondence Address: MICHAEL BEST & FRIEDRICH LLP, 100 E WISCONSIN AVENUE, Suite 3300, MILWAUKEE, WI, 53202, US

(21) Appl. No.: 12/250,849

(22) Filed: Oct. 14, 2008

Related U.S. Application Data
(63) Continuation-in-part of application No. 12/117,342, filed on May 8, 2008.
(60) Provisional application No. 60/998,478, filed on Oct. 11, 2007.
(60) Provisional application No. 60/998,477, filed on Oct. 11, 2007.

(51) Int. Cl. A61B 17/00; H01B 7/00

(52) U.S. Cl. 606/1; 174/70 R

(57) ABSTRACT

The present invention provides, among other things, means to suppress AC current propagation along elongated medical devices incorporating long conductive structures. AC currents in the frequency range from approximately 10 MHz to 3 GHz can be substantially suppressed without altering the low and DC frequency response of the medical device.

### RELATED APPLICATIONS

This application is a continuation-in-part of U.S. application Ser. No. 12/117,342, filed May 8, 2008, the contents of which are incorporated herein by reference. This application also claims the benefit of U.S. Provisional Application No. 60/998,478, filed Oct. 11, 2007, and 60/998,477, filed Oct. 11, 2007, the contents of both of which are incorporated herein by reference.

### FIELD OF THE INVENTION

The present invention relates to methods and devices for reducing or eliminating the effects of electromagnetic fields on long metallic structures as are typically found in medical devices such as leads, catheters, guide wires, needles and cannulars.

### BACKGROUND

Medical devices, including but not limited to guide wires, transseptal needles, cannulars, and the like, often employ conductive metals or alloys such as stainless steel, Nitinol, brass, carbon nanotubes and others in the form of solid rods or tubes because these materials have superior mechanical characteristics, including torque transfer and tensile strength. These rods and tubes present conductive surfaces that when exposed to electromagnetic fields, such as for example those present in magnetic resonance imaging (“MRI”) systems, may sustain undesired currents or voltages that interact with the surrounding blood and tissue, potentially resulting in unwanted tissue heating, nerve stimulation or other negative effects resulting in erroneous diagnosis or therapy delivery.

Other medical devices, including but not limited to electrocardiographs (“ECGs”), electroencephalographs (“EEGs”), squid magnetometers, implantable pacemakers, implantable cardioverter-defibrillators (“ICDs”), neurostimulators, electrophysiology (“EP”) mapping and radio frequency (“RF”) ablation systems, and the like, consist of or commonly employ one or more conductive surfaces, often in the form of leads and catheters that either receive or deliver voltage, current or other electromagnetic pulses from or to an organ or its surrounding tissue for diagnostic or therapeutic purposes.

Further, such structures commonly include bare or insulated, single or multi strand cables, rods and tubes or may include single or multi filar coils of bare or insulated wire or a combination of some or all of the above to facilitate the transfer of mechanical forces and/or the conduction of electrical signals to and from the proximal (system) end to the distal (patient) end of the device. When exposed to electromagnetic fields, such as for example those present in magnetic resonance imaging (“MRI”) systems, these conductive surfaces may sustain undesired currents and or voltages that interact with the surrounding blood and tissue, potentially resulting in unwanted tissue heating, nerve stimulation or other negative effects resulting in erroneous diagnosis or therapy delivery.

An example of a typical medical device incorporating conductive surfaces in the form of rods and tubes is shown in FIG. I. The guide wire A typically consists of an atraumatic tip C and a shaft D. The tip is typically made by coiling a thin flexible wire around a tapered shaft, resulting in a very bendable tip designed to prevent injury to the vascular structure through which the guide wire is advanced towards its intended target. The shaft is typically made from stainless steel or Nitinol since both materials have superior characteristics, including tensile strength and torque transfer capability, for a given diameter, ranging from 0.006″ to 0.039″. Once in place, the actual diagnostic or therapeutic device, such as a balloon catheter or a stent delivery tool, slides over the guide wire and is advanced “over the rail,” reducing the risk of vascular puncture by the diagnostic/therapeutic tool.

The atraumatic tip is connected to the guide wire shaft, a continuous tube or rod (D), through a transition region (B), predominately designed to facilitate a strong, smooth transition between the two pieces of the guide wire. The transition region may also be used to establish an electric connection between the typically conductive tip and the guide wire shaft, allowing the guide wire itself to be used as a diagnostic or therapeutic tool. The guide wire or sections thereof are sometimes covered with a thin insulating film (not shown in FIG. I) to isolate the shaft from its surroundings to, for example, maintain biocompatibility or provide electrical insulation for low frequency AC signals. The atraumatic tip and guide wire shaft can sometimes sustain currents when exposed to an electromagnetic field, such as for example, that encountered in an MRI system. These currents can, for example, induce heating or cause nerve stimulation in the tissue surrounding the device, either directly or by creating current pathways through direct contact points between the tissue and the atraumatic tip or the shaft.

Illustrations of multi stranded cables such as for example used for the transfer of diagnostic and therapeutic electromagnetic signals in ICD leads and RF ablation catheters are shown in FIGS. IIa and IIb. The cables E and K each consist of three (3) layers F, G, H and L, M, N of insulated and bare wires, respectively, twisted about the longitudinal axis. The cross-section of cable E is shown in FIG. IIc. In both cable examples, E and K, additional layers I and J are utilized to provide mechanical integrity, electrical layer-to-layer isolation or shielding, depending on the conductivity of the layer, or all of the above. The conductive paths provided by single or multi stranded wires can sustain unwanted currents when exposed to an electromagnetic field, such as for example encountered in an MRI system. These currents can induce heating in the tissue surrounding the device either directly or by creating current pathways through the tissue involving electrodes attached to cables.

FIG. IIIa shows a combination of multi stranded cables and a multi filar coil to transfer diagnostic and therapeutic electromagnetic signals to different electrodes of an ICD lead. The lead body consists of an insulating extrusion Q surrounded by an insulating jacketing material P. The extrusion Q contains various lumens to allow the cables R and coil S to be run through the extrusion. Coil S electrically connects a distal corkscrew shaped active fixation tip (helix) of the lead to the proximal pulse generator while at the same time allowing the transfer of torque from the proximal to the distal end during the implant procedure to facilitate the extension of the helix. In FIG. IIIb, the coil consists of four tightly bundled bare filars that are coiled at a certain, essentially constant pitch, resulting in a gap U between filar bundles. In FIG. IIIc, a smaller number of insulated filars is used, again coiled at a specific, essentially constant pitch, this time resulting in a gap Y between filar bundles. The coil consists of a set of filars. Even though the cables R are shown to be identical in FIG. IIIa, they may actually differ in diameter and construction since the electrical requirements for the cables connecting the shock electrodes to the pulse generator are more demanding than for the cable connecting the ring electrode to the pulse generator. The conductive paths provided by the cables and coil can sustain unwanted currents when exposed to an electromagnetic field, such as for example encountered in an MRI system. These currents can induce heating in the tissue surrounding the device either directly or by creating current pathways through the tissue involving electrodes attached to the cables and coil.

A typical approach to reduce the current and voltage induced in the catheter and lead-like structures is the use of discrete components, often self-resonating RF chokes or LC (“tank”) circuits to block RF currents on the wires or conductors. These components “break” or interrupt the original conductor, which may affect the mechanical characteristics of the device and increase the potential for mechanical failure, clearly making this approach impractical for devices, such as guide wires, that use tubes and rods for their tensile strength and torque transfer characteristics. In addition, discrete components such as capacitors and inductors cannot be obtained in small enough sizes to allow the manufacture of small diameter multi-stranded cables, in particular if multiple blocking circuits are required. Furthermore, a large current pulse is delivered through some of the cables in an ICD lead, placing an extra burden on the discrete component specifications, typically resulting in larger components not compatible with the lead space requirements.

### SUMMARY

In some embodiments, the present invention provides a medical device having one or more elongated bodies in the form of a rod or tube comprised of a base material such as stainless steel, Nitinol, brass, carbon nanotubes, etc., and having an electrical conductivity consistent with these materials. One or multiple coaxial layers of alternating conductivity materials, that is, resistive/dielectric layers followed by highly conductive layers, are formed on top of each other. One or more of these layers, in contrast to the base material, are not continuous, but rather consist of patterns that either by themselves, through interaction with other layers, the base material and/or the surrounding environment, either directly or through a dielectric/resistive layer, form electrical structures and barriers that are substantially different in their response to AC signals at one or multiple frequencies or frequency bands than that of a medical device formed from the base material alone. In some embodiments, the electrical structures are created to present high impedances or section of high impedances at a specific frequency or frequencies or frequency bands for AC signals propagating along the rod/tube. In other embodiments, the electrical structures are created to match the AC signal propagation properties of the rod/tube to its immediate environment, such as blood or tissue, at a specific frequency or frequencies or frequency bands.

In some embodiments, the electrical structures formed between one or more coaxial layers forms a string of inductors. In other embodiments, the structures form a string of low pass filters including shunt capacitances between one or more layers and series inductors formed on one or more layers. In some embodiments, the structures form parallel resonant circuits, formed by the shunt capacitance between various layers and series inductors on other layers. In some embodiments, a string of resonant circuits is created, either operating at the same frequency band or multiple frequency bands. In other embodiments, the electrical structures formed between one or more coaxial layers form a string of self-resonating inductors or a string of self-resonating inductors, either operating at the same frequency band or multiple frequency bands.

In some embodiments, two coaxial layers cover at least a lengthwise portion of at least one conductive shaft, the two coaxial layers including a first, inner layer and a second, outer layer, the first layer comprising a dielectric/resistive material (e.g., PEEK or PTFE) and the second layer comprising a highly conductive material (e.g., gold, silver, copper, or other metals). Furthermore, the second layer incorporates at least one or multiple patterns partially or fully exposing the first layer to form resistive, capacitive or inductive sections or combinations thereof.

Various embodiments herein suppress the propagation of alternating currents in the frequency range from approximately 10 MHz to 3 GHz.

In some embodiments, the present invention provides a medical device having one or more elongated bodies in the form of a multi stranded cable and in which at least one or more layers of the cable contain a set of wires of varying conductivities. The set of wires may include bare wires, insulated wires, non-conducting wires, wires of low conductivity, and wires of high conductivity. The set of wires is twisted along the longitudinal axis to form a part of the cable. The pitch of each layer may be adjusted as needed. The cable may also incorporate coaxially wrapped thin layers of foil or tubes of varying conductivity, providing a radial separation of the wire sets and the ability to control the electrical interaction between the wire sets. The cable may also include an insulating or conducting layer to provide mechanical stability and/or to control electrical interaction with the environment exterior to the cable.

In some embodiments, the present invention provides a medical device having one or more elongated bodies in the form of a multi stranded cable and in which at least one or more layers contain a set of wires of which at least one is a mechanically continuous wire including at least one or more insulated section and one or more non-insulated section. In addition, the set of wires may include bare wires, insulated wires, non-conducting wires, wires of low conductivity, and wires of high conductivity. The set of wires is twisted along the longitudinal axis to form a part of the cable. The pitch of each layer may be adjusted as needed. The cable may also incorporate coaxially wrapped thin layers of foil or tubes of varying conductivity, providing a radial separation of the wire sets and the ability to control the electrical interaction between the wire sets. The cable may also include an insulating or conducting layer to provide mechanical stability and/or control electrical interaction with the environment exterior to the cable.

In addition, the present invention provides a method of controlling the current induced by an electromagnetic field on a medical device including elongated conductive structures such as single or multi stranded cables. The method includes the act of forming a single inductor of desired inductance, a string of inductors with equal or different inductance, a single self resonant circuit between the layers of the cable, and a string of self-resonant circuits at a single or multiple frequencies between the layers of the cable, wherein the cable remains mechanically continuous. The method also includes the act of using the interaction between single or multi stranded cables in the elongated conductive structure of the medical device to suppress AC propagation at a specific frequency or frequencies or over a single or multiple frequency bands.

### BRIEF DESCRIPTION OF THE DRAWINGS

FIG. I is a perspective view of a typical medical device having elongated conductive pathways in the form of a tube or rod as typically found in guide wires.

FIG. IIa is a perspective view of a multi stranded cable utilizing insulated wires to form the layers of the cable. Such a cable can be found in RF ablation catheters.

FIG. IIb is a perspective view of a multi stranded cable utilizing bare wires to form the layers of the cable. Such a cable can be found in ICD leads.

FIG. IIc is a cross section of the cable shown in FIG. IIa.

FIG. IId is a cross section of the cable shown in FIG. IIa without the additional layer I.

FIG. IIe is a cross section of yet another multi stranded cable configuration.

FIG. IIIa shows a combination of multi stranded cables and a multi filar coil to transfer diagnostic and therapeutic electromagnetic signals to different electrodes of an ICD lead.

FIG. IIIb shows a coil consisting of tightly bundled bare filars.

FIG. IIIc shows a coil consisting of insulated filars.

FIG. 1 is a perspective view of a medical device incorporating a tube or rod with conductive surface pattern according to some embodiments of the present invention.

FIG. 1a is a longitudinal cross-sectional view of the area 10 marked in FIG. 1.

FIG. 1b is an equivalent electrical circuit diagram of the basic, core shaft D of FIG. I.

FIG. 1c is an equivalent electrical circuit diagram of the shaft modified according to some embodiments of the present invention.

FIG. 2 is a perspective view of a medical device incorporating a tube or rod with conductive surface pattern according to yet another embodiment of the present invention.

FIG. 2a is an equivalent electrical circuit diagram of the shaft modified according to yet another embodiment of the present invention.

FIG. 3 is a perspective view of a medical device incorporating a tube or rod with conductive surface pattern according to yet another embodiment of the present invention.

FIG. 4 is a perspective view of a multi stranded cable as used in medical devices in which one layer is formed according to some embodiments of the present invention.

FIG. 4a is an equivalent electrical circuit diagram of a cable formed according to some embodiments of the present invention.

FIG. 4b is a perspective view of the cable layer of FIG. 4 formed according to some embodiments of the present invention.

FIG. 4c is a perspective view of the wire set used to form the cable layer of FIG. 4b.

FIG. 4d is a wire used in the wire set of FIG. 4c. The wire is formed according to some embodiments of the present invention.

FIG. 5 is a perspective view of a multi stranded cable as used in medical devices in which one layer is formed according to yet another embodiment of the present invention.

FIG. 5a is a perspective view of the wire layer used in FIG. 5 according to some embodiments of the present invention.

FIG. 5b is a perspective view of the set of wires used to form the layer 27 of the multi stranded cable in FIG. 5 according to an embodiment of the present invention.

FIG. 6a is a perspective view of a coil formed by a multi filar wire set utilizing at least two different winding pitches over one or more sections of the coil according to some embodiments of the present invention.

FIG. 6b is a perspective view of a coil formed by a multi filar wire set with the insulation removed over one or more sections of the coil according to some embodiments of the present invention.

FIG. 6c is a perspective view of a coil formed with the multi filar wire set shown in FIG. 6d according to some embodiments of the present invention.

FIG. 6d is a perspective view of the multi filar wire set used to form the coil in FIG. 6c. The wire set utilizes wires including alternating insulated and bare wire sections created on a mechanically continuous wire according to some embodiments.

FIG. 6e is a perspective view of a coil formed with the multi filar wire set shown in FIG. 6f according to some embodiments of the present invention.

FIG. 6f is a perspective view of the multi filar wire set used to form the coil in FIG. 6e. The wire set utilizes wires including alternating insulated and conductive coating sections.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also, it is to be understood that phraseology and terminology used herein with reference to device or element orientation (such as, for example, terms like “central,” “upper,” “lower,” “front,” “rear,” “distal,” “proximal,” and the like) are only used to simplify description of the present invention, and do not alone indicate or imply that the device or element referred to must have a particular orientation. In addition, terms such as “first” and “second” are used herein for purposes of description and are not intended to indicate or imply relative importance or significance.

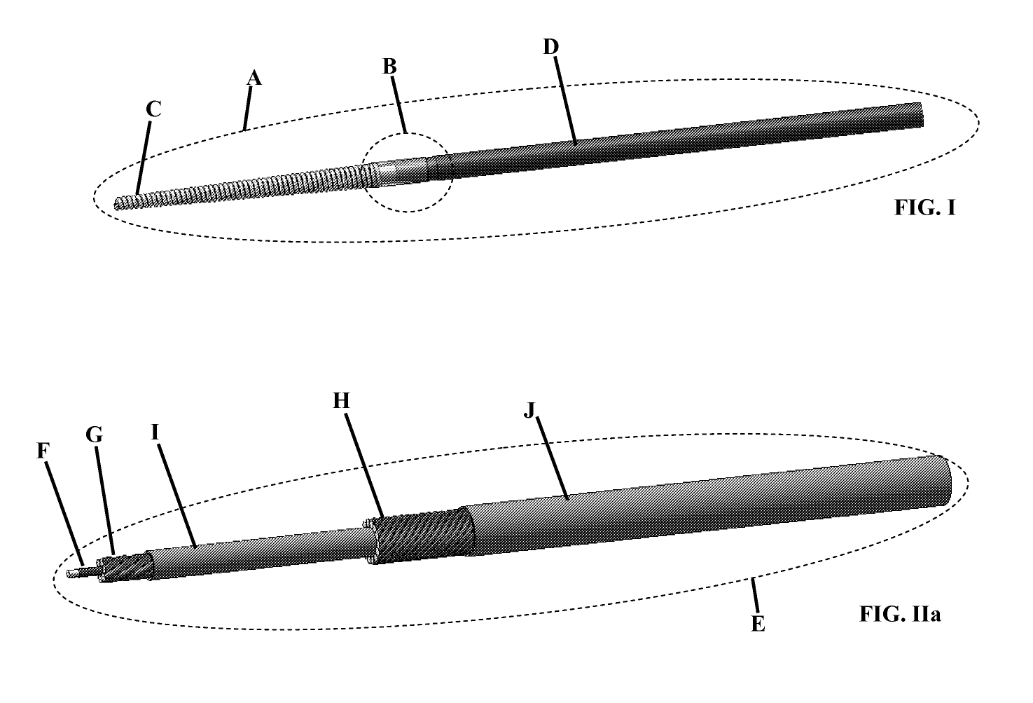

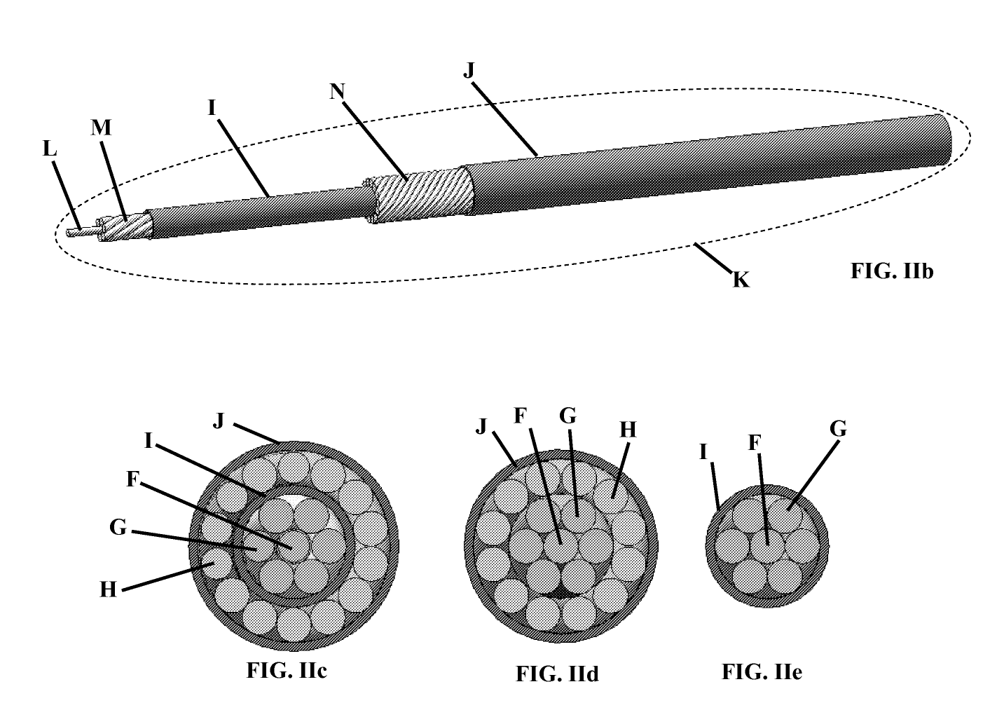

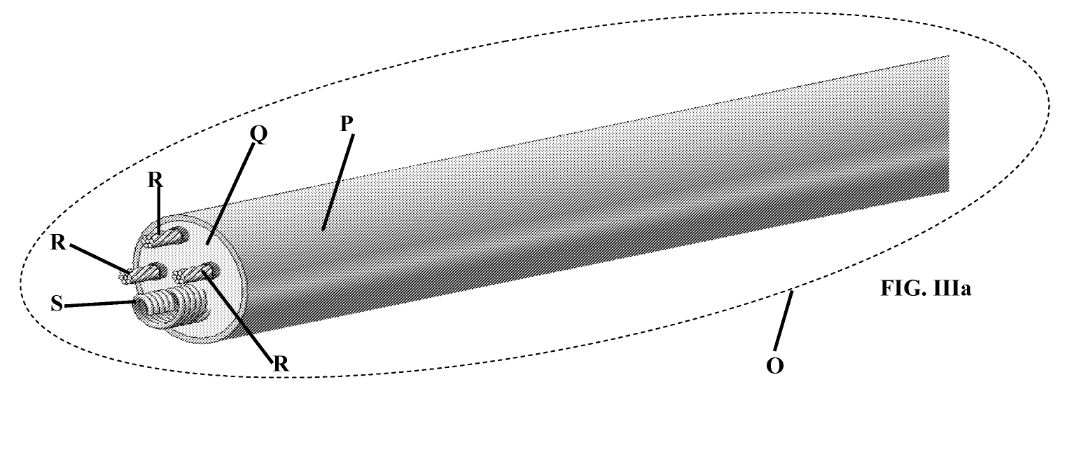

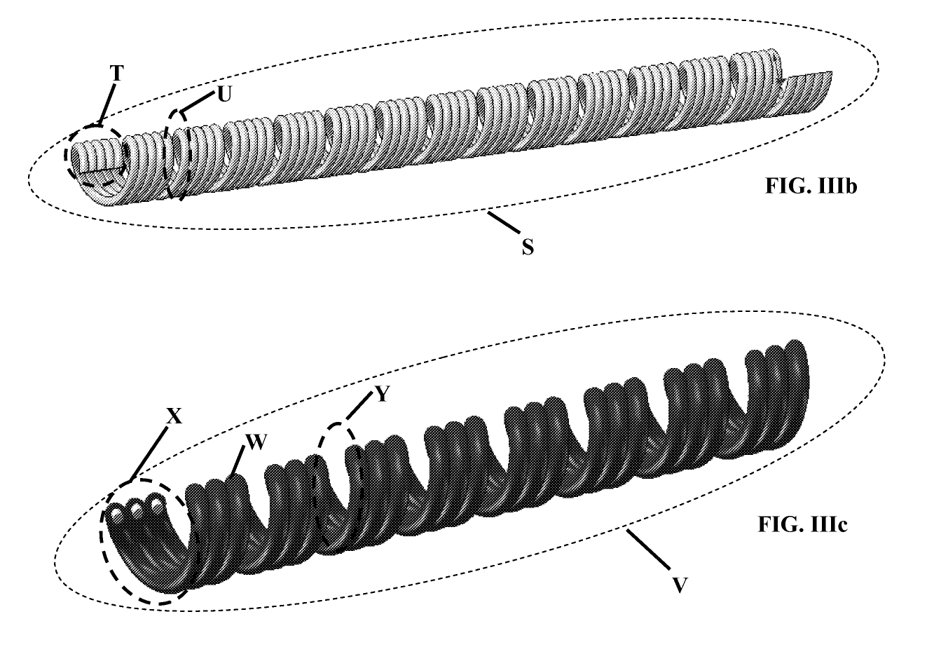

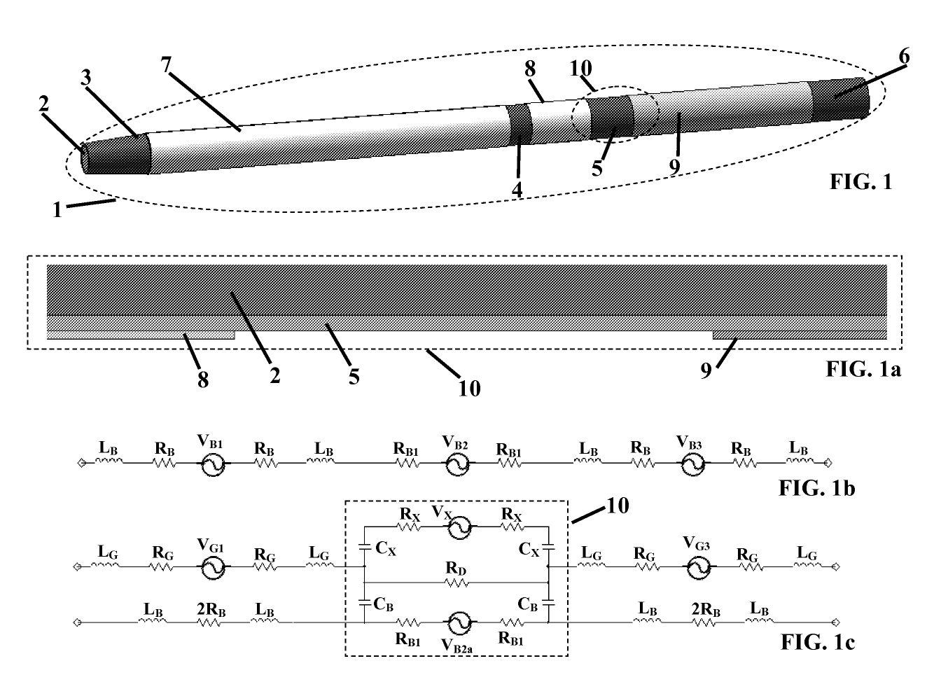

With reference to the Figures, a thin tube or rod, from hereon referred to as a shaft, according to the present invention is shown in FIG. 1. It will be understood by those of skill in the art that the shaft 1 could be part of any of a number of medical devices, including but not limited to guide wires, guide cannulars, EP mapping and ablation catheters, transseptal needles, etc.

In the embodiment of FIG. 1, a thin continuous dielectric layer is plated, extruded, “heat shrunk”, glued or in some other form deposited on a mechanically continuous shaft that could be made, for example, from Nitinol, stainless steel, brass or a carbon nano tube. The dielectric layer completely covers the shaft with the exception of a small area at the tip 2, allowing the transfer of low frequency signals through this area. A second, electrically conductive layer is plated or in some form deposited on the dielectric layer, for example as secondary tubing or tubing sections slipped and glued over the core/dielectric layer assembly; or for example as part of a dielectric/polymer material that has been “doped” in sections to be conductive. The purpose of the conductive layer is to force AC signal propagation on the top conductive surface and to fully or partially shield the core material. In contrast to the dielectric layer, the conductive layer contains patterns 7, 8 and 9, of varying length, thickness and/or conductivities, leaving sections 3, 4, 5 and 6 of the dielectric layer exposed.

The ability to form standing waves and the propagation efficiency of AC signals along the shaft surface at specific frequencies or frequency bands is significantly affected by the alternating conductive—dielectric material pattern. In some embodiments, a third, thin dielectric layer covers the outer surface to reduce the interaction with the surrounding material, either for electromagnetic reasons and/or to maintain biocompatibility. Optimized patterns for specific frequencies or frequency bands are determined by equivalent circuit analysis combined with computer simulations to determine circuit parameters such as capacitive coupling to the core material and if a third layer is present, capacitive coupling to the surrounding material. In some embodiments, the highest possible shaft impedance for AC signals is desired at specific frequencies or frequency bands, whereas in other embodiments a shaft impedance matching its surrounding material is more preferred.

As an example, the equivalent circuit for a standard shaft consisting only of the core material (“the core shaft”) is compared to a shaft constructed according to the embodiment shown in FIG. 1 with sections 7, 8 and 9 of equal length, thickness and conductivity (“the modified shaft”). Furthermore, for this example individual top layer conductive sections of the modified shaft are assumed to be short compared to the wavelength of interest. The voltage across each section can then be assumed to have a constant amplitude and phase, even though both amplitude and phase may vary between sections. As can be seen from FIG. 1a, capacitive coupling from the top conductive layers, here sections 8 and 9, to the core layer 2 is controlled by the thickness of the dielectric layer 5 and the length of the sections 8 and 9. Similarly, capacitive coupling to the external layer is controlled by the thickness of the third, top dielectric layer (not shown in the Figures).

The resulting equivalent circuit for the modified shaft representing area 10 of FIG. 1, including a third dielectric layer, is shown in FIG. 1c. Each top conductive section is represented by a series of inductors, resistors and voltage sources L_{G}, R_{G}, V_{G1} and V_{G3}. The gap 5 is represented by the resistance R_{D}; however, capacitive coupling to the core layer via the capacitors C_{B} and to the external layer via C_{X} create parallel conduction pathways, reducing the maximum achievable impedance. Taking into account phase shifts between the different conductive sections and the voltages induced in the external as well as the core layer, the AC propagation characteristic may be optimized further. The impedance of the core shaft over a section equivalent to the gap 5 is very small and can be approximated by the resistance R_{B1}. The modified shaft has substantially different AC propagation characteristics compared to the core shaft without degrading the mechanical characteristics of the continuous core material.

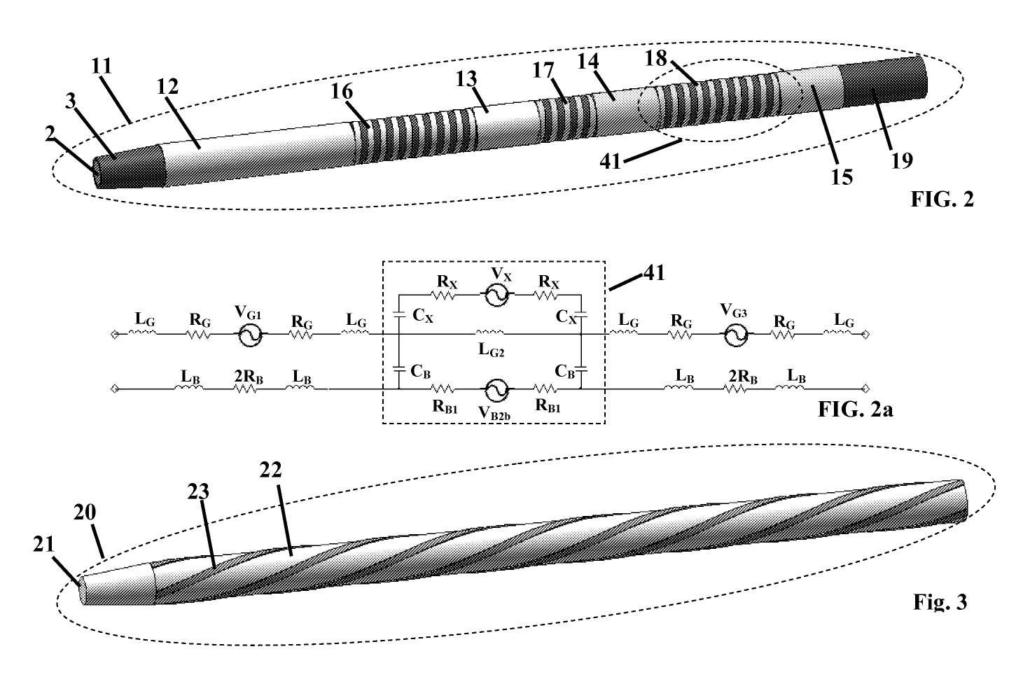

In a second embodiment, shown in FIG. 2, the top conductive layer has sections 12, 13, 14 and 15. In contrast to the embodiment shown in FIG. 1, the conductive sections are now connected via conductive patterns 16, 17 and 18, resembling solenoid inductors. An equivalent circuit for the shaft modified according to this embodiment, with sections 12, 13, 14 and 15 of equal length, thickness and conductivity, as well as sections 16, 17 and 18 of equal length, thickness, conductivity and turn density for the solenoids, is shown in FIG. 2a. The resistance R_{D} of FIG. 1c is now replaced by the inductor L_{G2}. This inductor and the capacitors C_{X} and C_{B} can be made to form a parallel resonant circuit, effectively suppressing AC current propagation along the shaft; or alternatively the AC propagation characteristics can be matched to the external material by appropriately selecting the capacitor ratios.

In some embodiments according to FIG. 2, the tip section of the shaft remains partially exposed, allowing the conduction of low frequency AC signals through the core as well as the top conductive layer. In other embodiments, the tip section can be covered by the dielectric material 5, preventing any low frequency propagation through the core of the shaft.

It will be apparent to those of skill in the art that other surface patterns can be created, such as for example the one shown FIG. 3, that allow the inductive, resistive and capacitive characteristics of the shaft to be manipulated to result in the desired AC impedance and propagation performance.

It will be apparent to those of skill in the art that fewer or additional layers can be used in the creation of the shaft 1. It will also be apparent to those of skill in the art that patterns can be created on more than one layer and that these overlaying patterns result in additional degrees of freedom to adjust the AC response of the resulting shaft.

Furthermore, it will be apparent to those skilled in the art that the order of material properties, such as conductivity and dielectric constant, can be reversed or arranged to result in more beneficial AC responses in different frequency bands.

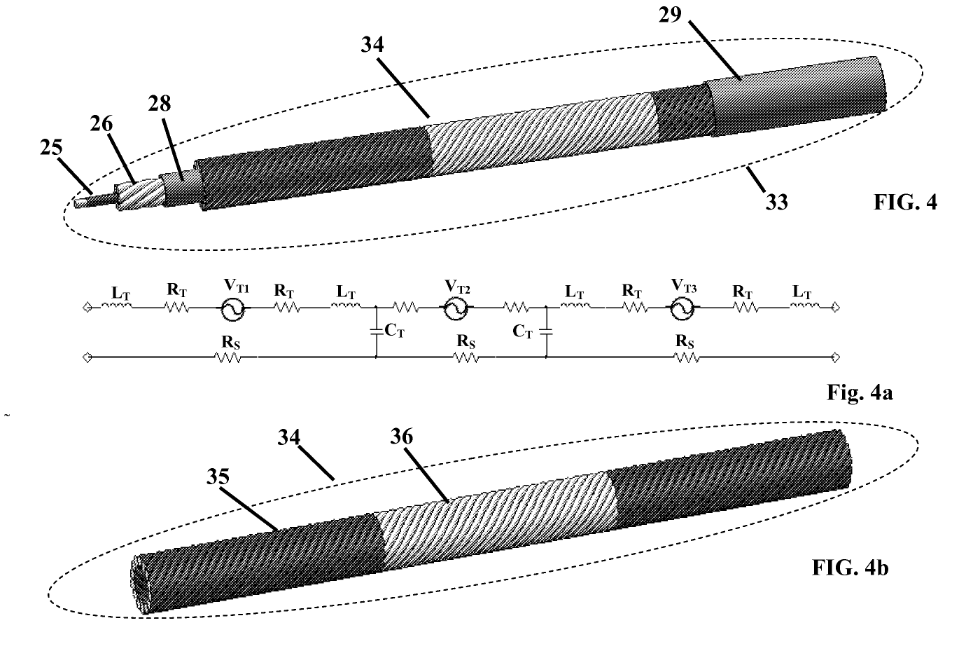

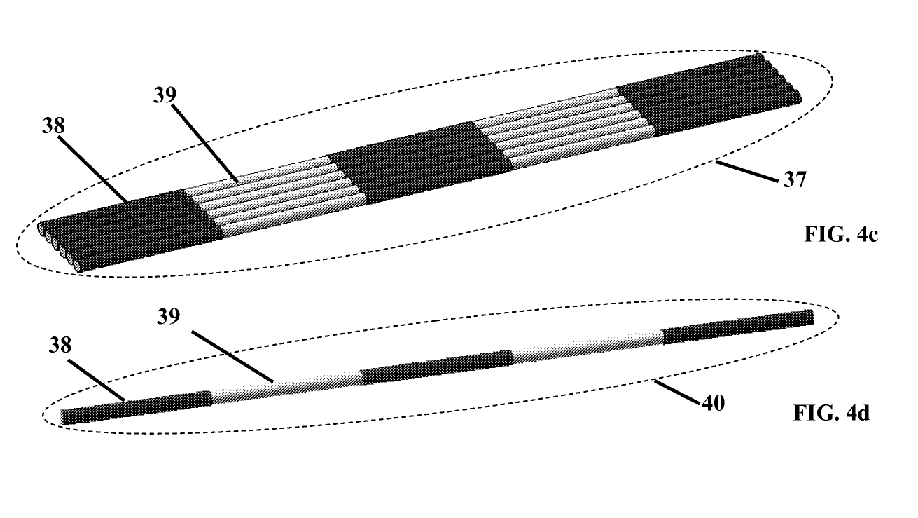

With reference to the Figures, a multi stranded cable, modified according to the present invention is shown in FIG. 4. It will be understood by those of skill in the art that the cable 33 could be incorporated in any of a number of medical devices, including EP mapping catheters, imaging catheters, RF ablation catheters, neurostimulator leads, ICD and pacemaker leads. The cable 33 consists of three conductor layers 25, 26 and 34 separated by insulating layers 28 and 29. The cable layer 34 in FIGS. 4 and 4b electrically presents a string of one or more inductors 35 connected via electrical short or low resistance section 36. The mechanically continuous cable layer 34 is formed by braiding (twisting) the wire set 37 of FIG. 4c around the longitudinal axis of the cable. The wire set 37 consists of single continuous wires 40 that, as shown in FIG. 4d, include insulated sections 38 and conductive sections 39. The conductive sections 39 either represent sections of bare wire and/or sections in which a conductive coating has been applied in some form over the sections of the wire. The latter approach allows the diameter of the conductive section to be manipulated to either be less than, equal to, or greater than the diameter of the insulated section. Because the wire 40 is mechanically continuous, the transition points between the insulated and non-insulated sections 35 and 36 of the cable layer 34 are mechanically continuous and do not require any means of joining such as soldering, welding, etc. It will be understood by those of skill in the art that the cable layer 34 of FIG. 4b could be comprised of more sections 35 and 36 or that the wire set 37 of FIG. 4c could include more or fewer wires 40, or that the wire set could include bare wires, or insulated wires or non-conductive wires or any combination thereof. It will also be understood by those of skill in the art that the cable 33 of FIG. 4 could have more or fewer layers and that one or more cable layers 34 could be used in the cable structure.

It will also be understood by those of skill in the art that the insulating layer 28 and 29 could be single insulating structures or could be double sided such that one side is conductive and the other is non-conductive or that one side contains patterns, such as for example described in the embodiments shown of FIGS. 1, 2 and 3.

In the embodiment shown in FIG. 4, the layer 26 consists of bare wire and is separated from the layer 34 via an insulating layer 28. The resulting equivalent circuit for this configuration is shown in FIG. 4a. The third layer is represented by a string of inductors, resistors and voltage source L_{T}, R_{T} and V_{T1} and V_{T3}, respectively, separated by a resistive section containing the voltage source V_{T2}. The sections are considered short such that the voltage source has constant amplitude and phase over the section at the wavelength of interests; however, amplitude and phase may vary from section to section. The bare wire section will primarily be responsible for the capacitive coupling C_{T} to the second layer. The second layer is to first order approximated by a string of resistive elements because the outer/third layer acts as a shield. If the shielding is insufficient, the insulating layer 28 can be modified to contain one conductive surface, in contact with layer 26, and one non-conductive surface, in contact with layer 34. The resulting equivalent circuit is shown in FIG. 4a and consists of series inductors joined across shunt capacitors; a typical low pass filter. The circuit can be transformed into a series of resonant LC circuits at specific frequencies or frequency band by appropriate choice of inductor and capacitor values, i.e., section length, dielectric constant and thickness of layer 28.

In some embodiments similar to that shown in FIG. 4, the insulation layer of the wire can be made very thin, for example, between 0.1 and 0.25 mil. This increases the turn-to-turn parasitic capacitance and effectively replaces the inductor L_{T} in FIG. 4a with a parallel LC circuit where the capacitance is distributed over the “inductor windings”. Choosing an appropriate pitch and section length, a resonant “tank” circuit is created, suppressing AC currents of the layer. Varying the pitch and length along the cable results in an AC current suppression at multiple frequencies or frequency bands.

In some embodiments, the alternating insulated and non-insulated sections 38 and 39 of the wire structure 40 are created by a removal process that removes partial sections from a fully insulated wire by chemical, mechanical, optical, or thermal means (e.g., chemical etching, mechanical grinding, laser burning, etc.). In other embodiments, the alternating insulated and non-insulated sections 38 and 39 of the wire structure 34 are created by a covering process that covers sections of a bare (non-insulated) wire with insulation material my means of partial extrusions, chemical deposition, etc. In yet other embodiments, the alternating insulated and non-insulated sections 38 and 39 of the wire structure 34 are created by a coating or extrusion process utilizing alternating or multiple types of coating/extrusion materials. These materials may include PTFE, PEEK, conductive polymers, etc.

In some embodiments, alternating insulated and non-insulated sections 35 and 36 of the structure 34 are formed by initially creating the structure using fully insulated wire and subsequently removing partial sections from the fully insulated section by chemical, mechanical, optical or thermal means. In other embodiments, the alternating insulated and non-insulated sections 35 and 36 of the structure 34 are formed initially from bare wire and sections are subsequently covered with insulation material by means of “dipping” or chemical deposition.

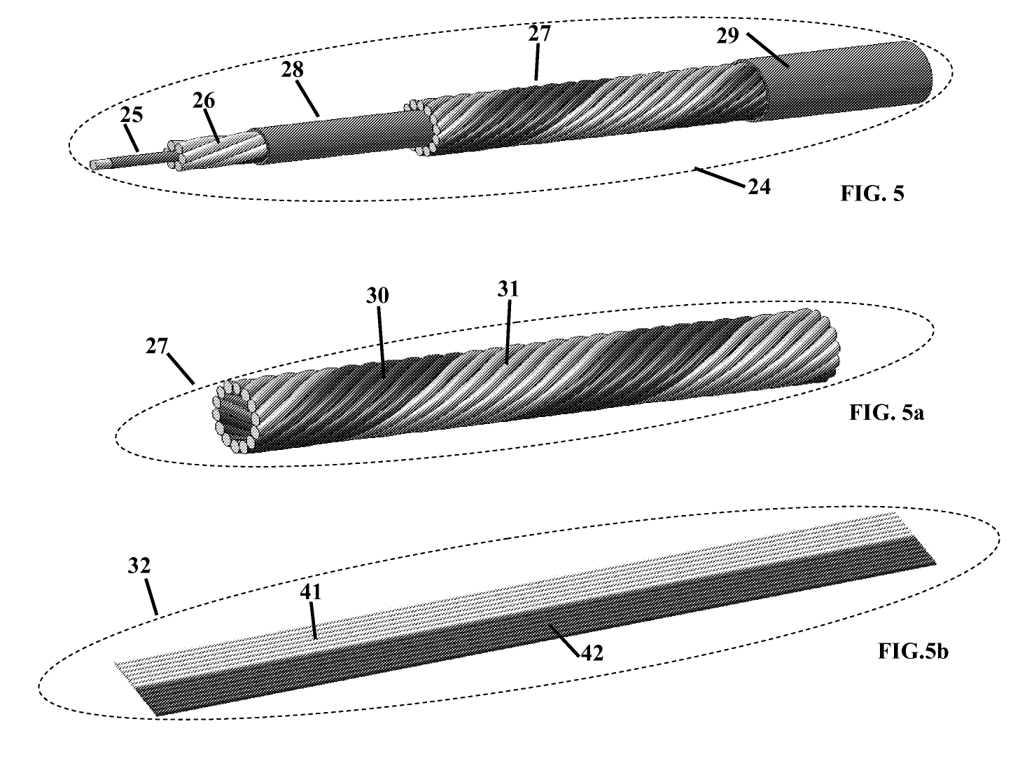

In yet another embodiment of the invention, shown in FIG. 5, the multi stranded cable 24 utilizes a third layer 27. The cable layer 27 in FIGS. 5 and 5a electrically presents a string of one or more inductors 30 connected via electrical short or low resistance section 31. The mechanically continuous cable layer 27 is formed by braiding (twisting) the wire set 32 of FIG. 5b around the longitudinal axis of the cable. The wire set 32 consists of mechanically continuous bare and insulated wires 41 and 42, respectively. Because the wires 41 and 42 are mechanically continuous, the transition points between the insulated and non-insulated sections 30 and 31 of the cable layer 27 are mechanically continuous and do not require any means of joining such as soldering, welding, etc. It will be understood by those of skill in the art that the cable layer 27 of FIG. 5a could be comprised of more sections 30 and 31 or that the wire set 32 of FIG. 5b could include more or fewer wires 41 or 42, or that the wire set could include non-conductive wires or wires of differing conductivities or any combination thereof. It will also be understood by those of skill in the art that the cable 24 of FIG. 5 could have more or fewer layers and that one or more cable layers 27 could be used or that other cable layers, such as 34 could be used in combination with layer 27 in the cable structure. At the lower end of the frequency spectrum (10 MHz to 3 GHz), it is advantageous to utilize thin wire insulation to increase the parasitic capacitance between the insulated windings and thereby increase the impedance of the insulated sections.

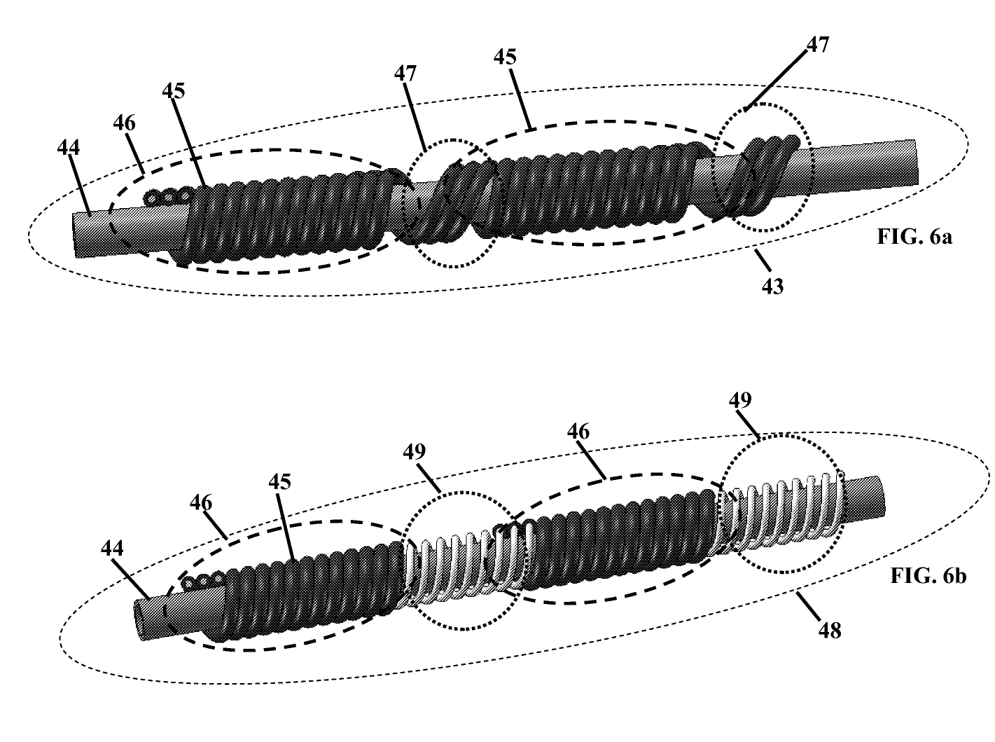

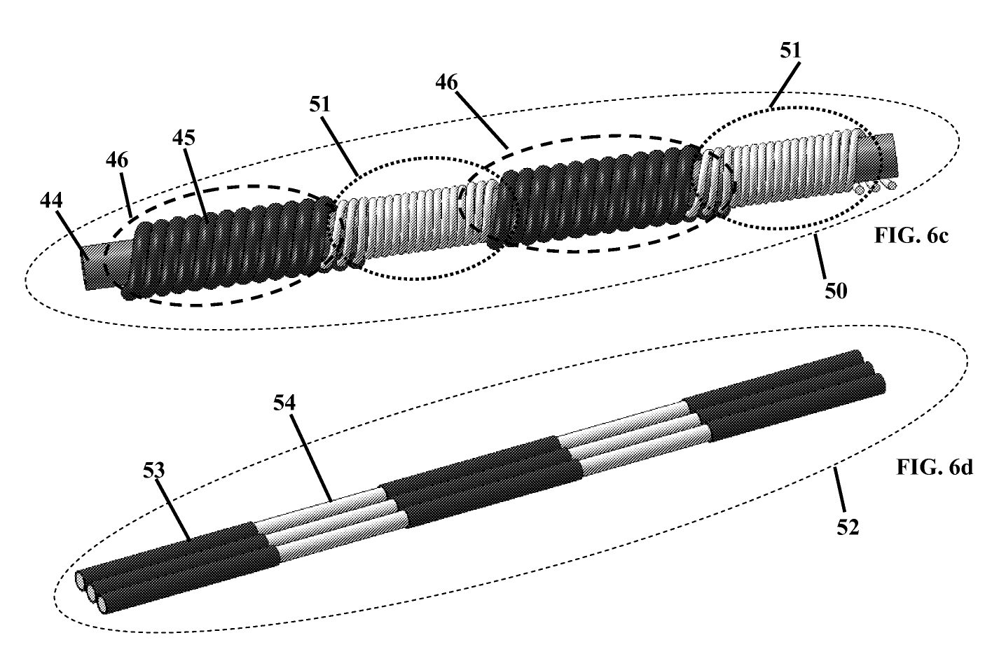

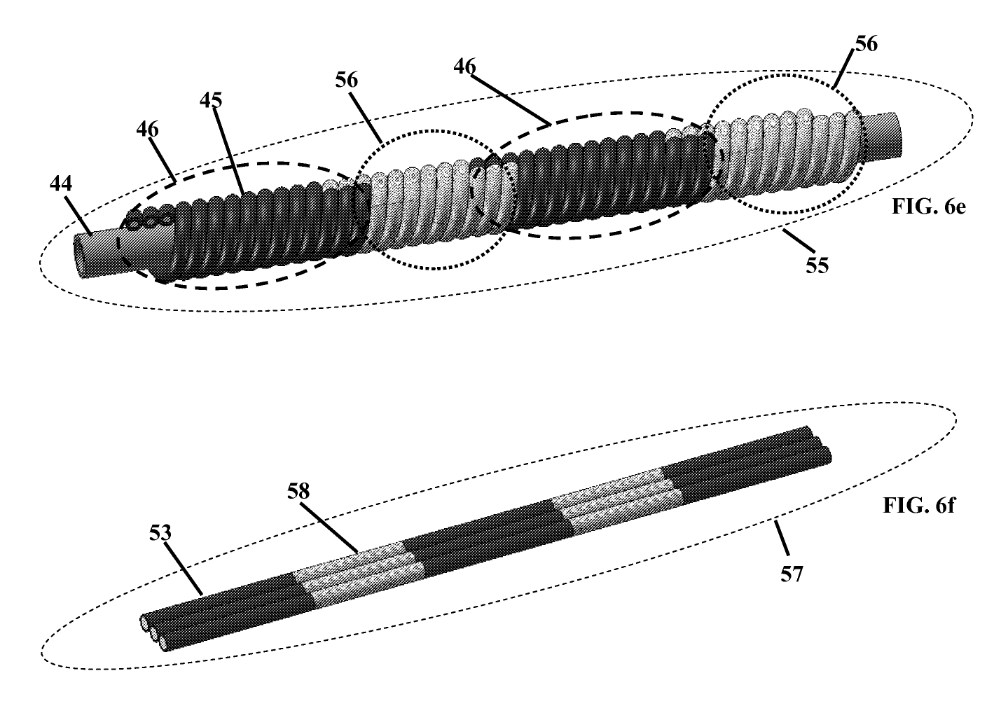

In yet another embodiment of the invention, shown in FIG. 6a, the coil(s) of pacemaker or ICD leads or other medical devices incorporating coiled wire to transfer diagnostic and therapeutic energy from the system end to the patient end are modified to form high impedance sections 46 by closely winding the insulated wire 45 coaxially along the lead body. The sections will behave as lumped elements as long as the coiled length is small compared to the wavelength at the frequency of interest. This is achieved by introducing a variable pitch, resulting in a gap 47. The impedance of section 46 can be increased compared to the impedance of an ideal inductor by adjusting the parasitic turn-to-turn capacitance by appropriate choice of the insulation thickness. Since the inductor section 46 forms a parallel LC circuit with the parasitic capacitance, it is possible to significantly increase the impedance; however, when the section becomes too long, the impedance will start to decrease and become capacitive. The precise behavior is controlled by varying the pitch over small sections. This approach essentially results in a string of high impedances joined by small inductive impedances.

In the embodiment shown in FIG. 6b, a constant pitch is maintained and the high impedance sections 46 are now joined by bare wire sections of the same pitch. The bare sections can be created, for example, by sand blasting a wire section and thereby removing the insulation locally.

In the embodiment of FIG. 6c, wire(s) including alternating insulated and bare wire sections (e.g., see FIG. 6d) are coiled along the lead body. The pitch is adjusted to result in a tightly wound coil consisting of insulated (inductor) and bare (short circuit) sections. The high impedance sections are now joined by node like sections. For large insulation thickness, a noticeable step down in diameter is observed as well as a change in pitch.

In the embodiment of FIG. 6e, wire(s) including alternating insulated and conductive sections (e.g., see FIG. 6f) are coiled along the lead body. The alternating sections are, for example, created via a coating or extrusion process in which the material is switched during the process. The resulting structure can be a string of high impedances joined by short circuit sections. In contrast to FIG. 6c, there now is full control over the coil diameter. The conductive sections now can be made to have a smaller, equal or larger diameter than the insulated sections. In some cases, it is useful to use hydrophilic material for the conductive sections since this can result in a swelling of these sections, forcing electrical turn-to-turn contact.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention as set forth in the appended claims.

What is claimed is:

1. A medical device, comprising: at least one conductive shaft, wherein one or more coaxial layers with one or multiple material deposition patterns cover at least a lengthwise portion of the at least one conductive shaft to suppress the propagation of alternating currents in the frequency range from approximately 10 MHz to 3 GHz.

2. The device of claim 1, wherein two coaxial layers cover at least a lengthwise portion of the at least one conductive shaft, the two coaxial layers including a first, inner layer and a second, outer layer, the first layer comprising a dielectric/resistive material and the second layer comprising a highly conductive material, wherein the second layer includes one or multiple patterns partially or fully exposing the first layer to form resistive, capacitive or inductive sections or combinations thereof.

3. The device of claim 3, further comprising a third, dielectric layer, the third layer (a) preventing direct contact of the second layer with an environment around the medical device, or (b) creating capacitive coupling of the one or multiple patterns of the second layer with the environment.

4. A medical device, comprising: at least one non-conductive shaft, wherein one or more coaxial layers with one or multiple material deposition patterns cover at least a lengthwise portion of the at least one non-conductive shaft to allow the propagation of DC and low frequency current therethrough while suppressing the propagation of alternating currents in the frequency range from approximately 10 MHz to 3 GHz.

5. The device of claim 4, wherein a first, coaxial layer covers the non-conductive shaft, the first layer comprising a conductive material and including one or multiple patterns partially exposing the shaft to form inductive sections.

6. The device of claim 5, further comprising a second, dielectric layer, the second layer (a) preventing direct contact of the first layer with the environment around the medical device, or (b) creating capacitive coupling of the one or multiple patterns of the first layer with the environment.

7. The device of claim 4, wherein a first coaxial layer covers the non-conductive shaft, the first coaxial layer comprising a conductive material, and a second and a third coaxial layer cover at least a lengthwise portion of the non-conductive shaft, the second layer comprising a dielectric/resistive material and the third layer comprising a highly conductive material, wherein the second layer incorporates one or multiple patterns partially or fully exposing the first layer to form resistive, capacitive or inductive sections or a combinations thereof.

8. The device of claim 7, further comprising a fourth, dielectric layer, the fourth layer (a) preventing direct contact of the third layer with the environment around the medical device, or (b) creating capacitive coupling of the one or multiple patterns of the second layer with the environment.

9. A medical device, comprising: one or more cables comprising one or more strands of wire, wherein the one or more strands of wire incorporate a pattern of insulated and conductive sections to suppress the propagation of alternating currents in the frequency range from approximately 10 MHz to 3 GHz.

10. The device of claim 9, comprising one or more coaxial layers of insulating material covering a lengthwise portion of the wire strands to control the capacitive coupling between the coaxial layers of stranded wire.

11. The device of claim 9, comprising one or more coaxial layers of insulating material partially or fully coated on one or both sides with a conductive substance covering a lengthwise portion of the wire strands to achieve one or more of the following:
(a) control inductive coupling between coaxial wire strands by partially or fully shielding the wire strands,
(b) control capacitive coupling between coaxial wire strands, and
(c) provide capacitive coupling to insulated sections of a wire strand.

12. The device of claim 9, wherein the material used for the insulated sections is sufficiently thin or of high dielectric constant to result in a substantial wire-to-wire capacitance to substantially modify the impedance of the insulated sections compared to that of an ideal inductor.

13. The device of claim 12, wherein the material thickness and/or dielectric material is chosen to result in a self-resonating section of high impedance near a target frequency within approximately the 10 MHz to 3 GHz range.

14. The device of claim 12, wherein the material thickness and/or dielectric material is chosen to result in an increased impedance near a target frequency within the approximately 10 MHz to 3 GHz range.

15. The device of claim 9, wherein the cable strands are formed utilizing continuous wires with alternating insulated and conductive sections.

16. The device of claim 15, wherein the alternating insulated and conductive sections are formed utilizing an extrusion or coating process switching between an insulating material and a conductive polymer.

17. The device of claim 16, wherein the insulating material is PTFE or PEEK.

18. The device of claim 16, wherein the thickness of the conductive and insulating sections is substantially identical.

19. The device of claim 15, wherein the conductive sections are bare wire.

20. The device of claim 9, wherein the cable strands are formed utilizing continuous wires of varying conductivities, including insulated wires, wires with conductive coating, and non-conductive wire/filars.

21. A medical device, comprising: one or more coils comprising one or more filars of continuous wire, wherein the one or more coils have one or more tightly wound sections to create at least one high impedance section, the at least one high impedance section having a length that is short compared to the wavelength at one or more frequencies or frequency bands of interest in the frequency range approximately 10 MHz to 3 GHz.

22. The device of claim 21, wherein a plurality of high impedance sections are created, the high impedance sections being separated by one or more variable pitch sections.

23. The device of claim 21, wherein a plurality of high impedance sections are created, the high impedance sections being separated by one or more bare wire sections created by sand blasting or ablating insulation of the wire, leaving a gap in inductor sections of the wire.

24. The device of claim 21, wherein a plurality of high impedance sections are created, the high impedance sections being separated by tightly wound bare wire sections utilizing one or multiple wires with one or more alternating bare and insulated sections.

25. The device of claim 21, wherein a plurality of high impedance sections are created, the high impedance sections being separated by tightly wound conductive wire sections utilizing one or multiple wires with one or more alternating conductive and insulated sections.

26. The device of claim 21, wherein the at least one high impedance section is inductive in the frequency range of interest.

27. The device of claim 21, wherein the at least one high impedance section is capacitive in the frequency range of interest.
